(19) United States
(12) Patent Application Publication
(10) Pub. No.: US 2013/0178885 A1
(43) Pub. Date: Jul. 11, 2013

(54) TROCAR SYSTEM FOR USE IN LAPAROSCOPIC SURGERY

(71) Applicant: Jeong Sam Lee, Gwangju (KR)

(72) Inventor: Jeong Sam Lee, Gwangju (KR)

(21) Appl. No.: 13/824,468

(22) PCT Filed: Jun. 24, 2011
(86) PCT No.: PCT/KR11/04621

§ 371 Date: Mar. 18, 2013

(30) Foreign Application Priority Data
Sep. 17, 2010 (KR) 10-2010-0091315
Jan. 24, 2011 (KR) 10-2011-0007001

(51) Int. Cl. A61B 17/34

(52) U.S. Cl. CPC A61B 17/34
USPC 606/185

(57) ABSTRACT

A trocar system for use in laparoscopic surgery is disclosed. According to an embodiment of the present invention, the trocar system includes: an upper housing having a circular or oval shape with a lower opening; a plurality of protrusion membranes provided to the upper housing to install a trocar; one or more tubes for sustaining air passage installed on the upper housing; and a lower guide formed at the lower edge of the upper housing, installed on the upper portion of a laparoscopic retractor, and conveniently removed therefrom. The trocar is fixedly installed on the protrusion membrane, and includes: a trocar body having a cylindrical shape; a valve cover disposed over the trocar body to close the upper space thereof; a recess for an installation ring disposed on the lower outer circumference of the trocar body; and an elastic press which is moved in conjunction with the side portion of an upper valve to open the upper valve; the trocar system further includes a seal part. When the trocar body is inserted into the protrusion membrane, the seal part is disposed at the recess for an installation ring to fix the outer circumference of the protrusion membrane.

### TECHNICAL FIELD

The present invention relates to a trocar system which helps insert a laparoscopic surgery instrument which is used for the sake of a laparoscopic surgery while making sure that an injury cut for surgery can remain sealed from the outside.

### BACKGROUND ART

Different from a conventional laparoscopic surgery, a new laparoscopic surgery is developed, which is likely to minimize the skins which will be cut, so the surgical operation in a laparoscopic surgery method increases day by day.

The above mentioned laparoscopic surgery has features in that multiple small holes are made at a patient's abdomen with the aid of trocars, and a surgery instrument such as an endoscope, etc. is inserted into a surgery portion of an abdomen, and the surgery is performed while monitoring the surgery portion of the abdomen through the inserted endoscope.

The laparoscopic surgery is widely used for the sake of a surgery operation such as a lap cholecystectomy, an extirpation of choledocal stone, an extirpation of intrahepatic gallstones, a gastrectomy, a colectomy, a small bowel resection operation, etc.

The trocar among many surgery instruments used for the sake of laparoscopic surgery has features in that an end portion of a trocar body is integrally extended to a channel having a certain size, while communicating with the channel, and an insertion part is formed at one end of the same for the sake of an insertion into the hole formed at the abdomen.

It is preferred that at least one work channel is provided so as to make sure that various instruments can pass so that tissues are observed and engaged, and the tissues are cured so as to perform a diagnosis or curing effect.

A hole is formed at the wall of the abdomen during the abdomen procedure in the laparoscopic surgery, and at least one tubular cannula or a retractor is inserted into the abdominal cavity (which is an empty space surrounded by the abdomen membranes of the abdomen below the abdominal diaphragm (horizontal membrane), and the upper side of the same is separated from the thoracic cage by means of the horizontal membrane, and the lower side of the same comes into contact with the pelvis, and the vertical portion is surrounded by the vertebral column and the abdominal muscles or other muscles).

In order to monitor the portions being under the surgery operation, a laparoscopic surgery-dedicated camera connected to the monitors of the operation room can be used. The above mentioned cameras might be arranged by way of one of the work channels.

Different laparoscopic surgery instruments such as a grasper, a dissector, scissors, a retractor, etc. might be arranged by way of at least one work channel so that a surgery doctor and/or surgery doctors assistants can easily perform various operations.

The laparoscopic surgery advances a lot, and it still has high potentials for the developments. The technology of the laparoscopic surgery by way of the navel is a key technology for the sake of the developments of the laparoscopic surgeries. The surgery operation by way of the navel is performed using a long trocar or is performed in a state that the cut window is maintained by way of the navel using variously formulated retractors.

The retractor is directed to a device for the purpose of obtaining a passage through which a laparoscopic surgery is performed at an injured cut-away window, and a trocar is engaged at the retractor for making easier the insertion of the surgical instrument.

In the above mentioned retractor, it takes much time for many equipment to be engaged or disengaged, and the manufacture costs a lot, so the price of the product increases.

Since the device for the sake of a passage removing smokes generating during the injection of the air and the surgery is engaged by way of the trocar, the whole volume of the trocar increases.

The long and tubular cannula-shaped trocar makes hard the insertion of the bent laparoscopic surgery instrument while being interfered with other surgical instruments.

In addition, the formulated retractor which is currently used might be used to obtain only a passage for the sake of a surgery in a fixed state, the operation of the instrument has a limit, and the construction of the product is complicated.

### DISCLOSURE OF INVENTION

Accordingly, it is an object of the present invention to provide a trocar system which can be engaged at or disengaged from the upper side of the retractor system used for the sake of the laparoscopic surgery and which makes easier the engagements of various laparoscopic surgery instruments.

It is another object of the present invention to provide an economical trocar system which is shorter and has a simplified construction for thereby engaging the trocars which can be easily sealed from the outer environment.

The trocar system for the use in laparoscopic surgery is disclosed.

According to one aspect of the present invention, there is provided a trocar system for use in a laparoscopic surgery, comprising a circular or elliptical upper housing the lower side of which is open; a protrusion membrane for engaging a plurality of trocars provided in the upper housing; at least one air passage tube engaged at the upper housing; and a lower guide which is formed at a lower rim of the upper housing and is detachably engaged at an upper side of a retractor used for the laparoscopic surgery injuries, and the trocar is fixedly inserted into the interior of the protrusion membrane for the sake of trocar engagements.

In addition, the trocar comprises a cylindrical trocar body; a valve cover which is provided at the upper side of the trocar body and seals the upper space; an engagement circular groove formed at a surrounding portion of the lower side of the trocar body; an elastic presser which opens the upper valve in sync with one side of the upper valve; and a seal part fixedly pressuring a surrounding portion of the protrusion membrane from the circular groove.

In addition, the upper side of the trocar body is formed in a slope surface structure which is inwardly inclined, and the lower side of the valve cover has a slope surface matching with the slope surface structure.

According to another aspect of the present invention, the trocar comprises a trocar body which is formed in a cylindrical shape, and a wall surface of one side is longer than a wall surface of the other side, thus providing an inclined lower space surface; a valve cover configured to cover the slope lower space surface, thus sealing the lower side; a circular groove for engagements formed at an outer surrounding portion of the upper side of the trocar body; an elastic presser which opens the lower valve in sync with one side of the lower valve cover; and a seal part fixing an outer surrounding portion of the protrusion membrane at the circular groove.

In addition, the elastic presser is formed in an outwardly bent arc shape, and as on side of the same operates in sync with the valve cover, when the outwardly bent arc shape portion is pressed, the valve cover operates in sync and is opened, and when the pressing force is eliminated, it is elastically closed.

According to further another aspect of the present invention, the trocar comprises a cylindrical trocar body; a wing shaped valve cover sealing the lower side of the trocar body by means of a plurality of elastic wings provided at the lower side of the trocar body; a circular groove for engagement formed at an outer surrounding portion of the trocar body; and a seal part fixing an outer surrounding portion of the protrusion membrane.

### ADVANTAGEOUS EFFECTS

The trocar system according to an embodiment of the present invention is advantages in that it can be separated from a retractor system, and the engagement is easy, and a simplified structure is provided, so the engagement and cost can be saved.

The trocar system according to an embodiment of the present invention has a short length and a smaller diameter, and various trocars which are easily sealed from the external environment can be provided, and the whole volume of the trocar system can be reduced.

The trocar system according to an embodiment of the present invention is advantageous in that a bent surgical instrument can be easily engaged, and a work radius between surgical instruments is wide, and the interruption and collision between surgical instruments can be prevented, and the whole surgery time can be reduced.

### BRIEF DESCRIPTION OF DRAWINGS

FIG. 1 is a plane view illustrating a trocar system according to the present invention.

FIG. 2 is a view illustrating a structure of a trocar system according to the present invention.

FIG. 3 is a plane view illustrating a trocar system according to another embodiment of the present invention.

FIG. 4 is a view illustrating a structure of a trocar system according to another embodiment of the present invention.

FIG. 5 is a cross sectional view illustrating a construction that a trocar system is engaged at a retractor according to the present invention.

FIG. 6 is a view illustrating a structure of a trocar according to a first embodiment of the present invention.

FIG. 7 is a view illustrating a construction that a valve cover of a trocar is sealed according to a first embodiment of the present invention.

FIG. 8 is a view illustrating a construction that a valve cover of a trocar is opened according to a first embodiment of the present invention.

FIG. 9 is a view illustrating a state that a trocar is engaged at a housing of a trocar system, and a surgical instrument is inserted according to a first embodiment of the present invention.

FIG. 10 is a view illustrating a state that a valve cover of a trocar is sealed according to a second embodiment of the present invention.

FIG. 11 is a view illustrating a state that a valve cover of a trocar is opened according to a second embodiment of the present invention.

FIG. 12 is a view illustrating a state that a trocar is engaged at a housing of a trocar system, and a surgical instrument is inserted according to a second embodiment of the present invention.

FIG. 13 is a view illustrating a structure of a trocar according to a third embodiment of the present invention.

FIG. 14 is a view illustrating a state that a trocar is engaged at a housing of a trocar system, and a surgical instrument is inserted according to a third embodiment of the present invention.

### DESCRIPTIONS OF REFERENCE NUMERALS

1: trocar system engagement elastic ring
2: trocar system upper housing
3, 4, 5, 6: trocars
7, 8: air, gas maintaining tube
14, 106, 206: trocar body
16, 102, 204: circular grooves
23, 116: valve cover
28, 120: elastic presser
30, 123, 218: seal part
32, 124, 219: upper housing protrusion membrane
100: laparoscopic surgery instrument
210, 217: wing type valve cover

### BEST MODES FOR CARRYING OUT THE INVENTION

The present invention has various modifications and embodiments, and the descriptions of the present invention will be described along with the embodiments with reference to the drawings, and it is not intended that the present invention is limited to specific embodiments, and it is interpreted that all the conversions, equivalents and substitutions belonging to the concept and technical scope of the present invention are included in the present invention.

During the descriptions of the invention, when it is seemed that the descriptions on the related prior arts make the subject matters of the invention unclear, the descriptions thereon will be omitted.

The terms “first” and “second” might be used for the purpose of describing various construction elements; however such elements should not be limited by the above terms, and the above terms are used for the purpose of separating one element from another element.

The terms used in the present invention is used for the sake of the descriptions of the specific embodiments, not limiting the present invention. A single form of expression is meant to include multiple elements unless otherwise stated. The term “comprise” or “include” in the present invention is directed to indicating the presences of features, numbers, steps, operation, elements, parts or a combination of the same, and should not be interpreted as eliminating the presences or addable possibilities of one or at least one feature, numbers, steps, operations, elements, parts or a combination of the same.

The illustrative embodiments will be described for the purpose of providing whole understandings with respect to the structure, function and use principles of the system disclosed in the present invention, and at least one example of the embodiments will be illustrating in the accompanying drawings,

The constructions detailed in the present specification and the structures shown in the drawings are provided for illustrative purposes for easier descriptions of the present invention, and the features illustrated or described in relation with the embodiments of the present invention might be combined with the features of other embodiments. Such modifications and changes belong to the scope of the present invention.

The embodiment of the present invention will be described in relation with the laparoscopic surgery procedures in the abdomen. The system might be used for the sake of human being or animal body or other surgical procedures. For example, the system disclosed according to an embodiment of the present invention might be used for a thoracic cage, a pelvic cavity, a cranial cavity or a certain natural hole of the human body or might be used for an endoscopic procedure or an abdomen surgical procedure.

The embodiments of the present invention will be described with reference to the accompanying drawings.

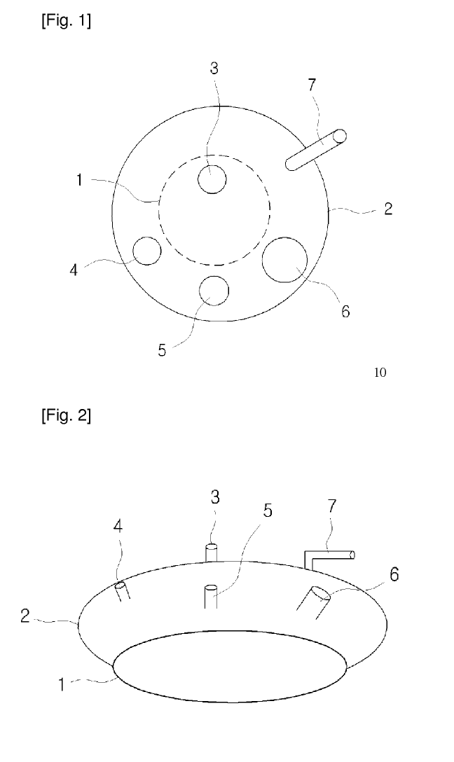

FIGS. 1 and 2 are a cross sectional view and a view illustrating the trocar system according to the present invention.

The trocar system according to the present invention has features in that a plurality of trocars 3, 4, 5 and 6 are engaged at the cylindrical or elliptical upper housing 2 the lower side of which is open.

The upper housing might be implemented in various shapes such as a cylindrical shape or an egg shape.

The trocar system of FIGS. 1 and 2 is formed of a plurality of engaged trocars 3, 4 and 5 each having an outer diameter of about 5.5 to 8 mm, and depending on the usage, a larger trocar 6 with a diameter of 10.5 to 13 mm can be engaged. (It is preferred that the outer diameter of the trocar according to an embodiment of the present invention is about 0.5 mm to 3 mm).

Considering that the minimum inner and outer diameters of the trocars are 0.5 mm, when the outer diameter becomes smaller than 5.5 mm, the engagement of 5 mm surgical instrument might be impossible, and when the diameter becomes greater than 8 mm, the space between trocars is limited, so the collision between 5 mm surgical instruments might occur.

Considering that the diameter of 10 mm surgical instrument is 10 mm, the engagement might become easier when the outer diameter of the trocars for the sake of 10 mm surgical instrument is at least above 10.5 m for easier engagement. If the diameter exceeds 13 mm, the collisions might occur between 10 mm surgical instruments.

The trocar according to the present invention is formed of a trocar having a short length of 4 to 40 mm (when adapted to a surgical instrument of a diameter of below 10 mm) or 8 to 40 mm (when adapted to a surgical instrument of a diameter of above 10 mm) for the sake of an easier insertion of a long laparoscopic surgical instrument or a bent laparoscopic surgical instrument.

If the length of the trocar becomes below 4 mm, the engagement of the elastic presser for the same of the operation of the elastic cover according to an embodiment of the present invention might be impossible or the handling of the same might be hard. When the length becomes above 40 mm, the insertion of the bent surgical instrument might be hard, and the work radius between the surgical instruments might be small, and the collisions between the surgical instrument might occur.

According to a result of the test performed by engaging a surgical instrument at the trocar system according to the present invention, a problem did not occur in the surgical work up to the length of the trocar of 60 mm; however as the diameter and length of the trocar become longer, the surgical section between the surgical instruments is limited, and the leftward and rightward movements of the surgical instrument are limited, and the collision might occur.

Compared to the conventional trocar having a diameter of 30-50 mm and a length of 100 to 200 mm, the trocar system according to the present invention has a relatively smaller diameter and a shorter length, so the insertion of the bent surgical instrument can be possible, and the work radius of the surgical instruments becomes wider, and it is possible to prevent the interruptions and collisions between the surgical instruments.

An air passage tube or a smoke elimination passage 7 might be engaged at the upper housing 2.

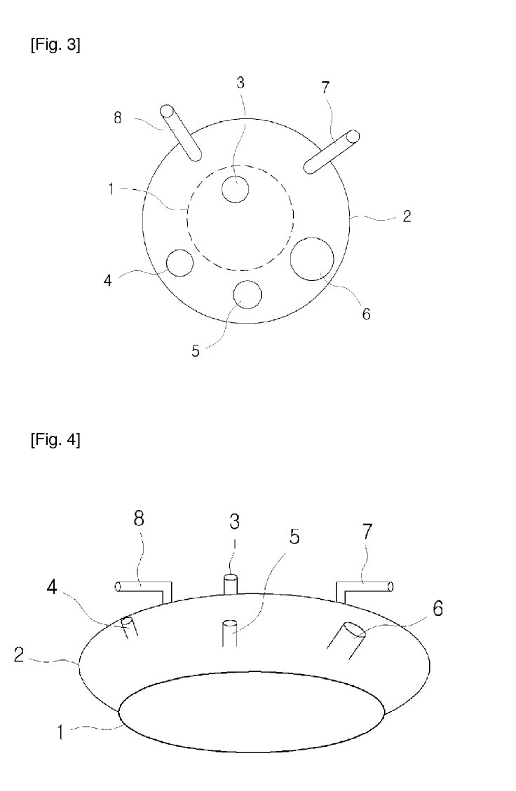

As shown in FIGS. 3 and 4, in another embodiment of the present invention, there is further provided an injection tube or passage 8 for injecting carbon dioxide in addition to the air passage 7.

The upper housing 2 of the trocar system according to the present invention might be formed of a polymer containing a polycarbonate and a polyetheretherketone, a metal such as titanium or stainless steel, a composite like a carbon-fiber-reinforced PEEK, a ceramic material and a combination of the same.

It might be formed of a semi-strength/flexible material including a thermoplastic elastic polymer such as polyurethane, a polyisoprene polymer, a silicon polymer latex of a middle to high hardness and a combination of the same.

In the embodiment of the present invention, the upper housing is made from a material such as a silicon elastic polymer latex, etc. having flexibility. If the upper housing is made from a flexible material, the interval between the trocars can be flexibly adjusted, so it is possible to prevent the collision between the surgical instruments or the interference between them during the surgical operation as compared to when forming it with a hard material fixing the trocars or the instrument passages.

At the lower side of the trocar system according to an embodiment of the present invention is provided a lower guide helping an easier attachment and detachment at the upper side of the laparoscopic surgery retractor.

A flexible material is used for the lower guide, and an elastic ring 1 is provided at the rim for the elastic installation at the upper side of the retractor system 300.

The elastic ring 1 is made from a good elastic material for the purpose of stably fixing the trocar system.

In addition, cylindrical protrusion membranes 32, 124 and 219 are provided at the upper side of the upper housing 2 for the purpose of fixedly engaging the trocars as shown in FIGS. 9, 12 and 14.

The cylindrical protrusion membranes 32, 124 and 219 are made from flexible materials or good elastic materials for the elongation and engagement works.

The body of the trocar is inserted into the interior of the cylindrical protrusion membrane, and the trocar can be fixedly engaged at the upper housing 2 by means of the seal parts 30, 123 and 218 as shown in FIGS. 9, 12 and 14 which strongly pressurize and fix the outer side of the protrusion membrane.

The seal parts 30, 123 and 218 might be fixed by tightening an elastic ring having a high elasticity.

It might be engaged using a thermal adhesion or a bond in an engaged structure at the time of the initial manufacture.

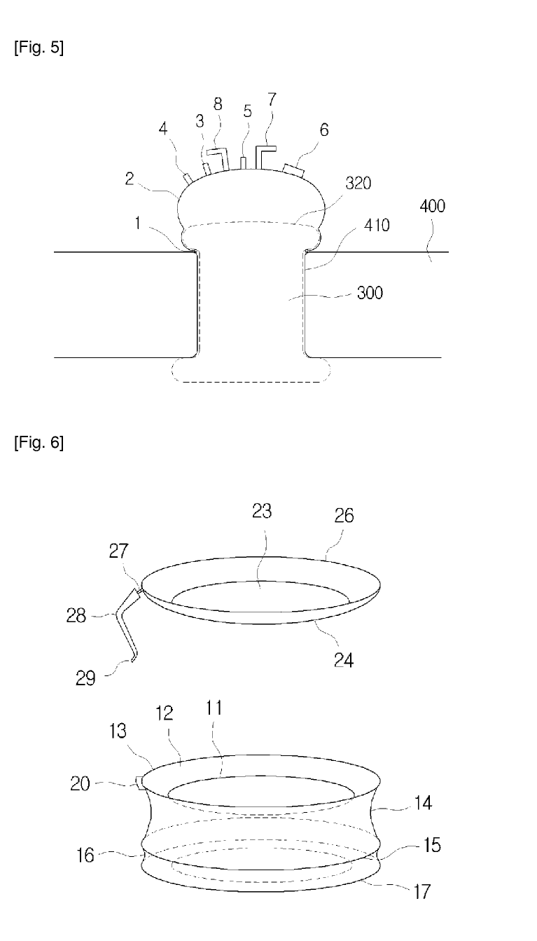

The trocar system according to the present invention is directed to a separated system for the sake of an easier separation engagement with the retractor system and is engaged at the upper guide 320 of the retractor system as shown in FIG. 5.

FIG. 5 is a cross sectional view illustrating a state that a trocar system is engaged in a retractor system according to the embodiment of the present invention.

The trocar system according to the embodiment of the present invention is invented to well fit the separation type retractor system.

First of all, when the retractor system the upper side of which is open is installed at the surgical cut part 410 of the tissue layer 400 such as a navel or the like, the elastic ring 1 of the lower guide of the trocar system is prolonged and is covered on the upper guide 320 of the retractor system, so the trocar system can be fixed at the retractor system.

The elastic ring 1 of the lower guide is smaller than the diameter of the upper ring 320 of the retractor system 300, and the engagement is finished as the elastic ring 1 is prolonged and covered on the upper ring 320, so the engagement and disengagement can be performed in an easier way.

Thanks to the separation and installation, the complicated mechanical structure can be simplified, so the whole volume of the laparoscopic surgery system can be reduced.

FIGS. 6 to 9 are views illustrating the structures of the trocars according to a first embodiment of the present invention.

The trocar body 14 according to the first embodiment of the present invention, it is made from a material elastic enough to closely push and insert the laparoscopic surgery instrument 100 and is formed in a cylindrical shape.

At the upper side of the trocar body 14 is provided a slope surface which is inwardly inclined to be sealed by the valve cover 23, and at one upper side of the slope surface is provided a hinge part 20.

The valve cover 23 is engaged in a structure which operates in sync with the elastic presser 28 and the connection part 27 about the hinge part 20.

At the lower side of the valve cover 23 is provided a cover slope surface 24 corresponding to the slope surface 24 of the trocar body, so it can be easily sealed as the valve cover 23 is closed.

The elastic presser 23 is bent in an angle structure shape like an outwardly bent arc shape, and the lower side 29 of the elastic presser 28 is bent in the reverse direction and is engaged so that it can be positioned at the protrusion shoulder 15 formed at the wall body of the trocar body.

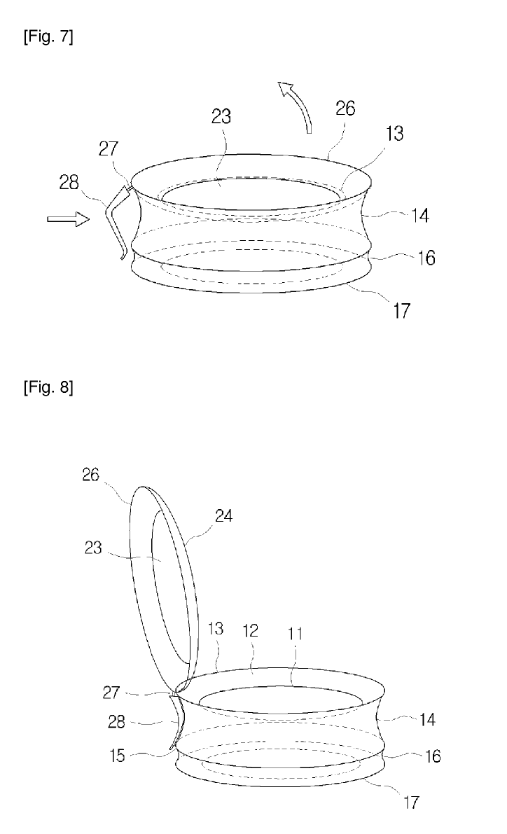

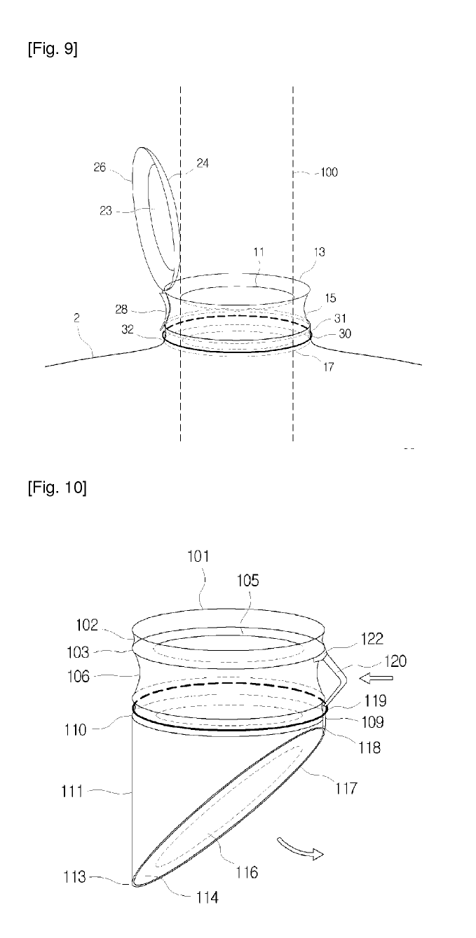

When the elastic presser 28 is pressed in the direction of the arrow as shown in FIG. 7 so as to install the laparoscopic surgery instrument 100, the lower side of the elastic presser 28 slides along the protrusion shoulder 15, so the bent portions go elastically straightened, and the valve cover 24 as shown in FIG. 8 is opened in sync with the above operation.

In addition, when the laparoscopic surgery instrument 100 is withdrawn, the elastic presser 23 go elastically straightened to its original shape, and the valve cover 23 is automatically closed.

FIG. 9 is a view illustrating a state that the trocar is engaged at the housing of the trocar system, and the surgical instrument is inserted according to a first embodiment of the present invention.

At the upper housing 2 according to the embodiment of the present invention is provided the cylindrical protrusion membrane 32 having a flexibility so as to fixedly engage the trocars at the upper side.

As shown in FIG. 9, at the lower side of the trocar is provided circular groove 16, and when the trocar body is inserted into the interior of the cylindrical protrusion membrane, the seal part 30 strongly and fixedly pressurizes the surrounding portions of the cylindrical protrusion membrane covering the circular groove.

The seal part 30 might be formed of a circular elastic ring having a high elasticity or a screw clamp clamping the clamping strand using the screws.

FIGS. 10, 11 and 12 are views illustrating the structures according to a second embodiment of the present invention.

The trocar body 106 according to the second embodiment of the present invention is made from a material elastic enough to closely push and insert the laparoscopic surgery instrument 100 and is formed in a cylindrical structure.

The cylindrical structure of the trocar body 106 has a surface 111 in which the wall surface of one side is longer than the wall surface of the other side.

In other words, the wall surface of one side is formed longer than the wall surface of the other side, so an end portion of one side of the slope lower surface 113 is connected with an end portion of one side of the valve cover 117 by means of the hinge part 118, whereby the valve cover 116 seals the inclined lower space.

The valve cover 116 is installed in a structure which operates in sync with the elastic presser 120 and the connection part 119 about the hinge part 118.

The elastic presser 120 is bent in an angle structure shape like an outwardly bent arc shape, and the upper side of the elastic presser 120 is fixed by means of the clamp 122.

At the upper side of the hinge part 118 is formed a slide groove 110, and at the slide groove 110 is installed a fixing ring 109 supporting the lower side of the elastic presser 120.

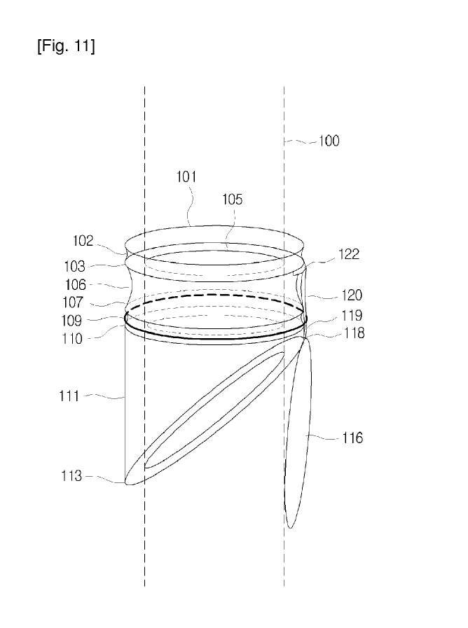

When the elastic presser 120 is pressed in the direction of the arrow as shown in FIG. 10 so as to install the laparoscopic surgery instrument 100, the lower side of the elastic presser 120 slides along the fixing ring 109, and the bent portions go elastically straightened, thus pushing the connection portion 119, so the valve cover 116 operating in sync with the same is opened as shown in FIG. 11.

When the laparoscopic surgery instrument 100 is withdrawn, the elastic presser 120 elastically goes back to its original state, and the valve cover 116 is automatically closed.

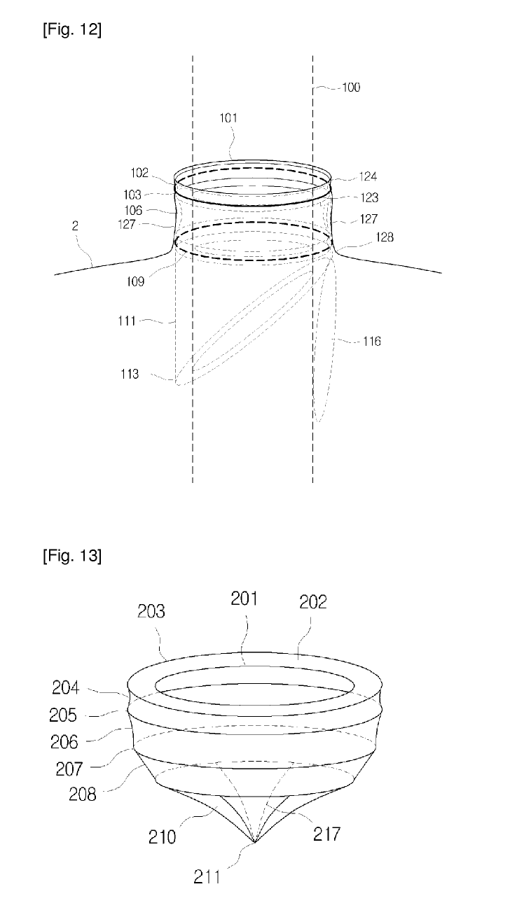

FIG. 12 is a view illustrating a state that the trocar is engaged at the housing of the trocar system, and the surgical instrument is inserted according to a second embodiment of the present invention.

As shown in FIG. 12, at the upper side of the trocar is provided a circular groove 102, and when the trocar body is inserted into the interior of the protrusion membrane 124 formed at the upper housing 2, the seal part 123 strongly fixes the surrounding portions of the protrusion membrane covering the circular groove 102.

The seal part 123 can be fixed by tightening with the elastic ring with a high elasticity or it can be engaged using a thermal adhesion or a bond in an engaged shape from the first stage.

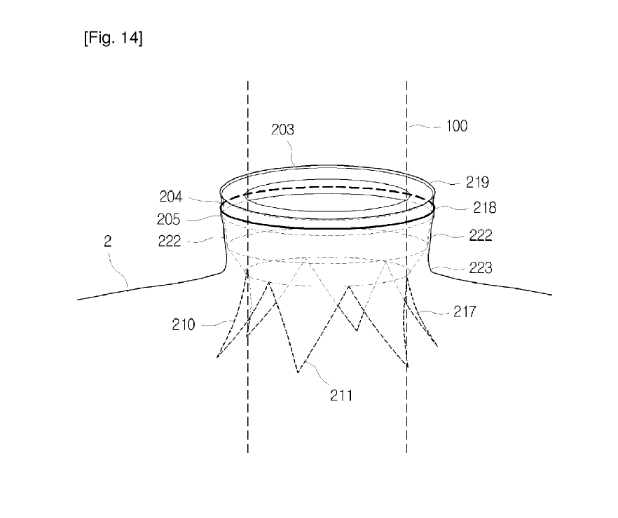

FIGS. 13 and 14 are views illustrating a construction of a trocar according to a third embodiment of the present invention.

The trocar body 206 according to a third embodiment of the present invention is made from a material elastic enough to closely push and insert the laparoscopic surgery instrument 100 and is formed in a cylindrical shape.

The cylindrical structure of the trocar body 206 has wing shape valve covers 210 and 217 sealing the lower openings with the aid of a plurality of elastic wings at the lower side.

As shown in FIG. 13, the wing shaped valve covers 210 and 217 have features in that the elastic wings are formed in an inverted triangle shape and are gradually narrowed in the downward direction and come into contact with each other at the lower side apex 211 and are elastically closed and sealed.

When the laparoscopic surgery instrument 100 is pushed in as shown in FIG. 14, the wing shaped valve covers 210 and 217 are divided and opened by the pushing force. When the laparoscopic surgery instrument 100 is withdrawn, the valve covers 210 and 217 are puckered again by an elastic force, thus sealing the lower side of the trocar body 206.

As shown in FIG. 14, at the upper side of the trocar is provided a circular groove 204. When the trocar body 206 is inserted into the interior of the protrusion membranes 222 and 219 formed at the upper housing 2, the surrounding portions of the protrusion membrane covering the circular groove 204 are strongly fixed by the seal art 218. Alternatively, it might be provided a structure engaged using a thermal adhesion and a bond when it is first engaged.

The trocar system according to the embodiments of the present invention has features in that various types of trocars can be engaged at the upper housing of the trocar system with the aid of the separable structure.

The present invention is made for the uses in the single passage surgical operation or the surgical operation by way of the navel; however it can be adapted to more delicate surgical operations such as a cranial cavity surgical operation, etc. because the size or the like can be easily adjusted.

The trocar and the trocar system according to the embodiment of the present invention has simplified structures, and the changes are easy depending on the sizes, so they can be well adapted to various surgical instruments which become compact-sized.

In addition, the trocar system according to an embodiment of the present invention has advantages in that the engagement separated from the retractor system is easy, and the structure is simplified, so the time and cost for the sake of the engagement of equipment can be saved.

The trocar system according to an embodiment of the present invention has a short length and a small diameter and a simplified structure, and various trocars which can be easily sealed from the outside can be engaged, and the whole volume with respect to the trocar system can be reduced.

In addition, the trocar system according to the present invention helps easily engage the bent surgical instrument, and the collision during the surgical operation is prevented, and the whole surgical operation time can be reduced.

### INDUSTRIAL APPLICABILITY

The trocar system according to the present invention can be adapted to the surgical operation.

The trocar system according to the present invention has features in that the laparoscopic surgery used for the sake of the laparoscopic surgical operation can be inserted and can be used for the purpose of maintaining the sealed state from the external environment with respect to the portions cut for the sake of the surgical operations.

1. A trocar system for use in a laparoscopic surgery, comprising:
a circular or elliptical upper housing the lower side of which is open;
a protrusion membrane for engaging a plurality of trocars provided in the upper housing;
at least one air passage tube engaged at the upper housing; and
a lower guide which is formed at a lower rim of the upper housing and is detachably engaged at an upper side of a retractor used for the laparoscopic surgery injuries.

2. The system according to claim 1, wherein the upper housing and the lower guide are made from a silicon elastic polymer having flexibility or a latex material.

3. The system according to claim 1, wherein a circular elastic ring is proved at the lower guide.

4. The system according to claim 1, wherein a trocar is fixedly inserted into the interior of the protrusion membrane for the trocar engagement.

5. The system according to claim 4, wherein the trocar has an outer diameter of 5.5 to 8 mm and a length of 4 to 40 mm.

6. The system according to claim 4, wherein the trocar has an outer diameter of 10.5 to 13 mm and a length of 8 to 40 mm.

7. The system of according to claim 4, wherein the trocar comprises:
a cylindrical trocar body;
a valve cover which is provided at the upper side of the trocar body and seals the upper space;
an engagement circular groove formed at a surrounding portion of the lower side of the trocar body;
an elastic presser which opens the upper valve in sync with one side of the upper valve; and
a seal part fixedly pressuring a surrounding portion of the protrusion membrane from the circular groove.

8. The system according to claim 7, wherein the upper side of the trocar body is formed in a slope surface structure which is inwardly inclined, and the lower side of the valve cover has a slope surface matching with the slope surface structure.

9. The system according to claim 4, wherein the trocar comprises:
a trocar body which is formed in a cylindrical shape, and a wall surface of one side is longer than a wall surface of the other side, thus providing an inclined lower space surface;
a valve cover configured to cover the slope lower space surface, thus sealing the lower side;
a circular groove for engagements formed at an outer surrounding portion of the upper side of the trocar body;
an elastic presser which opens the lower valve in sync with one side of the lower valve cover; and
a seal part fixing an outer surrounding portion of the protrusion membrane at the circular groove.

10. The system according to claim 7, wherein the elastic presser is formed in an outwardly bent arc shape, and as on side of the same operates in sync with the valve cover, when the outwardly bent arc shape portion is pressed, the valve cover operates in sync and is opened, and when the pressing force is eliminated, it is elastically closed.

11. The system according to claim 4, wherein the trocar comprises:
a cylindrical trocar body;
a wing shaped valve cover sealing the lower side of the trocar body by means of a plurality of elastic wings provided at the lower side of the trocar body;
a circular groove for engagement formed at an outer surrounding portion of the trocar body; and
a seal part fixing an outer surrounding portion of the protrusion membrane.

12. The system according to claim 11, wherein the wing shaped valve is elastically opened by the force generating when inserting the surgical instrument, and when the surgical instrument is eliminated, it is elastically closed.

13. The system according to claim 7, wherein the seal part is formed of an elastic ring.

14. The system according to claim 7, wherein the seal part is engaged by a thermal adhesive or a bond.

15. The system according to claim 9, wherein the elastic presser is formed in an outwardly bent arc shape, and as on side of the same operates in sync with the valve cover, when the outwardly bent arc shape portion is pressed, the valve cover operates in sync and is opened, and when the pressing force is eliminated, it is elastically closed.

16. The system according to claim 9, wherein the seal part is formed of an elastic ring.

17. The system according to claim 11, wherein the seal part is formed of an elastic ring.

18. The system according to claim 9, wherein the seal part is engaged by a thermal adhesive or a bond.

19. The system according to claim 11, wherein the seal part is engaged by a thermal adhesive or a bond.
